(19) United States
(12) Patent Application Publication
(10) Pub. No.: US 2025/0375229 A1
(43) Pub. Date: Dec. 11, 2025

(54) Smart Trauma System

(71) Applicant: Stryker European Operations Limited, Carrigtwohill (IE)

(72) Inventors: Veera Raju Vanapalli, Bengaluru (IN); Sunny Shorabh, Ghaziabad (IN); Rajan Yadav, New Delhi (IN); Denis Digeser, Freiburg (DE); Alexis Christen, Herzogenbuchsee (CH); Rachana K, Chennai (IN); Harpreet Singh, Bangalore (IN)

(21) Appl. No.: 19/226,958

(22) Filed: Jun. 3, 2025

Related U.S. Application Data
(60) Provisional application No. 63/657,460, filed on Jun. 7, 2024.

(51) Int. Cl. A61B 17/80; A61B 90/00

(52) U.S. Cl. CPC A61B 17/8014; A61B 90/06; A61B 2090/064

(57) ABSTRACT

A fixation system includes a bone plate configured to be placed against a bone; a first bone screw insertable through a hole in the bone plate, the bone screw including a sensor hermetically sealed within the bone screw and configured to detect a characteristic of at least one of the system and the bone; a power source configured to provide power to the sensor; and a processor configured to output the characteristic to an external source.

### CROSS-REFERENCE TO RELATED APPLICATIONS

This application claims the benefit of the filing date of U.S. Provisional Patent Application No. 63/657,460 filed Jun. 7, 2024, the disclosure of which is hereby incorporated herein by reference in its entirety.

### FIELD OF INVENTION

The present disclosure relates to medical implant devices, and particularly to bone plates and methods for monitoring bone conditions surrounding an implanted bone plate.

### BACKGROUND OF THE INVENTION

Many orthopedic procedures involving fractured bones require a bone plate that is positioned against the bone such that it spans multiple bone fragments. Various bone screws may be inserted through holes of the bone plate and driven into the underlying bone such that the plate stabilizes the bone and promotes healing.

Monitoring bone conditions during surgical procedures and while a patient recovers from various surgical procedures is critical for proper patient rehabilitation. A key component of monitoring a patient's recovery is detecting implant movement, subsidence, breakage, etc. Early identification of improper implant functioning and/or infection and inflammation at the implantation site can lead to corrective treatment solutions prior to implant failure. Data relating to postoperative range implant movement can be critical for managing recovery and identification of a proper replacement solution if necessary.

However, diagnostic techniques to evaluate bone conditions during and after surgeries are generally limited to patient feedback and imaging modalities such as X-ray fluoroscopy or magnetic resonance imaging (“MRI”). Patient feedback can be misleading in some instances. For example, bone plates and bone screws may wear or move over time, which may be imperceptible to a patient. Further, imaging modalities offer only limited insight into implant performance. For example, X-ray images will not reveal information related to the amount of stress on the patient's bone while recovering from a surgery. Furthermore, the imaging modalities may provide only an instantaneous snapshot of the bone conditions, and therefore fail to provide continuous real time information related to bone condition.

Therefore, there exists a need for improved bone plates and related methods for tracking bone conditions over time.

### BRIEF SUMMARY OF THE INVENTION

Disclosed herein are bone plate systems and methods for monitoring bone conditions surrounding implanted bone plates over a period of time.

According to one aspect of the present disclosure, a fixation system comprises: a bone plate configured to be placed against a bone; a first bone screw insertable through a hole in the bone plate, the bone screw including a sensor hermetically sealed within the bone screw and configured to detect a characteristic of at least one of the system and the bone; a power source configured to provide power to the sensor; and a processor configured to output the characteristic to an external source.

In another aspect, the system further comprises a second bone screw insertable through a second hole in the bone plate having a second sensor hermetically sealed within the second bone screw, the second sensor configured to detect a second characteristic of at least one of the bone and the bone screw.

In a different aspect, the system further comprises a wire sensor positioned between the first and second bone screws, the wire sensor configured to communicate with the processor to detect a third characteristic of at least one of the bone and the first and second bone screws.

In another aspect, the power source is mounted within at least one of the first and second bone screws.

In yet another aspect, the first and second bone screws are configured to be secured on opposite sides of a fracture line of the bone.

In a further aspect, the system further comprises a third bone screw insertable through a third hole in the bone plate, wherein the power source is mountable within a body of the third screw.

In a different aspect, the power source is a battery.

In another aspect, the power source is an external device capable of providing near field communication or power to at least one of the sensor and the processor.

In yet another aspect, the sensor is at least one of a strain gauge, accelerometer, gyroscope, temperature sensor, piezoelectric sensor, hall sensor, micro-electro-mechanical systems sensor, ultrasonic sensor, and pH sensor.

In a different aspect, the characteristic is at least one of a vibrational measurement of the screw, force between the bone plate and the bone, micromotions between a screw and plate, deflection between two screws due to load bearing, deformation of a plate due to load bearing, temperature, acceleration, and friction of an interface defined between the bone and the system.

According to another aspect of the present disclosure, a method of securing a bone, the method comprises placing a bone plate adjacent the bone; inserting a bone screw through a hole in the bone plate such that a sensor associated with the bone screw is configured to detect a characteristic; and outputting information of the characteristic to an external source.

In a different aspect, the method further comprises placing the sensor within a shank of the bone screw.

In another aspect, the method further comprises driving a second bone screw through a second hole in the bone plate.

In yet another aspect, the method further comprises powering the sensor with a battery housed within the second bone screw.

In a different aspect, the method further comprises powering the sensor with an external source, the external source capable of providing near field communication to the sensor and the processor.

In another aspect, the method further comprises storing the characteristic in a memory.

In a further aspect, the outputting step includes a processor receiving the characteristic and converting it to a characteristic signature, the characteristic signature being at least one of a vibration data, movement data, force data, strain data, and temperature data.

According to another aspect of the present disclosure, a fracture fixation system comprises a bone plate configured to be placed against a bone; a bone screw insertable through a hole in the bone plate, the bone screw associated with a processor; and a pressure sensor configured to be positioned on at least one of an upper and lower side of the bone plate, the pressure sensor in communication with the processor and configured to detect pressure profiles of at least one of the bone and the system such that the processor can receive the pressure profiles and output the pressure profiles to an external source.

In another aspect, the pressure sensor is a piezoelectric pressure sensor.

In a different aspect, the pressure sensor communicates with the processor by wireless communication.

In another aspect, the wireless communication is Bluetooth.

In a further aspect, the processor is housed within a hermetically-sealed cavity of the bone screw.

In another aspect, the system further comprises a second bone screw insertable through a hole in the bone plate, wherein the second bone screw includes a battery hermetically sealed within a cavity of the second bone screw, the battery configured to power the pressure sensor.

According to another aspect of the present disclosure, a method of securing a fractured bone, the method comprises placing a bone plate adjacent a bone; driving a bone screw associated with a processor through a first hole in the bone plate; creating, with the processor, a pressure profile based on a difference in pressure detected over a period of time by a pressure sensor positioned adjacent the bone plate; and sending the pressure profile to an external source.

In another aspect, the creating step includes creating the pressure profile based on a difference in pressure detected between the bone plate and the bone.

In a different aspect, the method further comprises placing a hermetic seal over the pressure sensor.

In yet another aspect, the sending step includes sending a second pressure profile to the external source.

In a different aspect, the method further comprises comparing the first pressure profile to the second pressure profile.

In a further aspect, the method further comprises creating an alert if a difference between the first and second pressure profiles exceeds a predetermined value.

In another aspect, the method comprises at least partially wrapping a pressure sensor around a fracture site and sensing hoop stresses that occur due to bone healing.

According to another aspect of the present disclosure, a fixation system comprises a bone plate configured to be placed against a bone; a first bone screw insertable through a first hole in the bone plate such that the first screw is positioned in the bone, the first bone screw including transmitter configured to output an ultrasonic wave; a second bone screw insertable through a second hole in the bone plate such that the second screw is positioned in the bone, the second bone screw including a receiver configured to receive an ultrasonic wave; and a processor configured to detect a change in ultrasonic energy between the transmitter and the receiver.

In another aspect, the transmitter is at least one of an ultrasonic sensor and electromagnetic sensor.

In a different aspect, the system further comprises a power source capable of powering the transmitter and the receiver.

In another aspect, the power source is an external device capable of communicating with near field communication.

In yet another aspect, the system further comprises a processor configured to compare a sent ultrasonic profile sent from the transmitter with a received ultrasonic profile received from the receiver.

In a different aspect, the processor is configured to communicate a difference in received ultrasonic profile to an external device.

In yet another aspect, the processor is configured to communicate the difference in received ultrasonic profiles over regular intervals of time.

According to another aspect of the present disclosure, a method of securing a bone, the method comprises placing a bone plate adjacent the bone; driving a first bone screw through a first hole in the bone plate and into the bone; driving a second bone screw through a second hole in the bone plate into the bone; and measuring a difference in ultrasonic energy detected between a transmitter positioned in the first bone screw and configured to output an ultrasonic wave and a receiver positioned in the second bone screw and configured to receive the ultrasonic wave.

In another aspect, the placing step includes placing the bone plate adjacent the bone such that the bone plate spans across a fraction line of the bone.

In a different aspect, the driving steps include driving the first bone screw into the bone on a first side of the fracture line and driving the second bone screw into the bone on a second side of the fracture line.

In a further aspect, the measuring step includes passing the ultrasonic wave through the fracture line of the bone.

According to another aspect of the present disclosure, a fixation system comprises a bone plate configured to be placed against a bone; a bone plate sensor positionable against the bone plate, the bone plate sensor configured to detect at least one parameter of the bone plate and the bone; a power source configured to provide power to the bone plate sensor; and a processor configured to store and output the parameter to an external source.

In another aspect, the bone plate sensor is positionable between the bone plate and the bone.

In a different aspect, the bone plate sensor is positionable on an outer surface of the bone plate.

In a further aspect, the at least one of the power source and the processor are hermetically sealed within the bone plate sensor.

In a different aspect, the system further comprises a first bone screw insertable through a first hole of the bone plate.

In another aspect, the at least one of the power source and the processor are hermetically sealed within the first bone screw.

In yet another aspect, the bone plate sensor is positionable within a groove of the bone plate.

In a different aspect, the bone plate sensor includes a first bone plate sensor configured to detect a first stage of bone healing and a second bone plate sensor configured to detect a second stage of bone healing.

In another aspect, the processor is configured to determine a condition of a patient's recovery using artificial intelligence.

### BRIEF DESCRIPTION OF THE DRAWINGS

A more complete appreciation of the subject matter of the present disclosure and the various advantages thereof can be realized by reference to the following detailed description, in which reference is made to the following accompanying drawings:

FIG. 1 is a perspective view of a bone plate system according to an embodiment of the present disclosure.

FIG. 2A is a side cross-sectional view of a bone screw implemented with the bone plate system of FIG. 1.

FIG. 2B is a side cross-sectional view of another bone screw implemented with the bone plate system of FIG. 1.

FIG. 2C is a side cross-sectional view of the bone screw of FIG. 2A implemented with a bone plate system.

FIG. 3 is a perspective view of a bone plate system according to another embodiment of the present disclosure.

FIG. 4A is a side view of a bone plate system according to another embodiment of the present disclosure.

FIG. 4B is a perspective view of the bone plate system of FIG. 4A.

FIG. 4C is another perspective view of the bone plate system of FIG. 4A.

FIG. 5 is a perspective view of a foil sensor implementable with the system of FIG. 4.

FIG. 6 is a top view of a diaphragm sensor implementable with the system of FIG. 4.

FIG. 7 is a side view of a bone plate system according to another embodiment of the present disclosure.

FIG. 8A is a side view of a bone plate system according to another embodiment of the present disclosure.

FIG. 8B is an enlarged side cross-sectional view of a portion of the bone plate system of FIG. 8A.

FIG. 8C is an enlarged perspective view of a portion of the bone plate system of FIG. 8A.

FIG. 8D is a side cross-sectional view of the bone plate system of FIG. 8A implementing a different sensor.

FIG. 8E is a side cross-sectional view of the bone plate system of FIG. 8A implementing a different sensor.

FIG. 8F is another side view of the bone plate system of FIG. 8A.

FIG. 9A is a side view of a smart bone screw inserted into a bone.

FIG. 9B is a side view of smart bone screws inserted through a bone plate and into a bone.

FIG. 9C is a side view of a smart bone screw acting as a syndesmosis screw inserted into a bone.

FIG. 10A is a perspective view of a bone plate system according to another embodiment.

FIG. 10B is another perspective view of the bone plate system of FIG. 10A.

FIG. 11 is a schematic flowchart of the logic of the bone plate system of FIG. 1

FIG. 12A is a schematic flowchart of a first AI system. FIG. 12B is a schematic flowchart of a training model implementable with the first AI system of FIG. 12A.

FIG. 12C is a schematic flowchart of another training model implementable with the first AI system of FIG. 12A.

FIG. 13A is a schematic flowchart of a second AI system.

FIG. 13B is a schematic flowchart of a training model implementable with the second AI system of FIG. 13A.

FIG. 14A is a schematic flowchart of a third AI system according to an embodiment.

FIG. 14B is a schematic flowchart of a third AI system according to another embodiment.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features within a different series of numbers (e.g., 100-series, 200-series, etc.). It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

In describing preferred embodiments of the disclosure, reference will be made to directional nomenclature used in describing the human body. It is noted that this nomenclature is used only for convenience and that it is not intended to be limiting with respect to the scope of the present disclosure. As used herein, when referring to bones or other parts of the body, the term “anterior” means toward the front part of the body or the face, and the term “posterior” means toward the back of the body. The term “medial” means toward the midline of the body, and the term “lateral” means away from the midline of the body. The term “superior” means closer to the head, and the term “inferior” means more distant from the head. The term “proximal” means closer to an operator, and the term “distal” means further away from an operator.

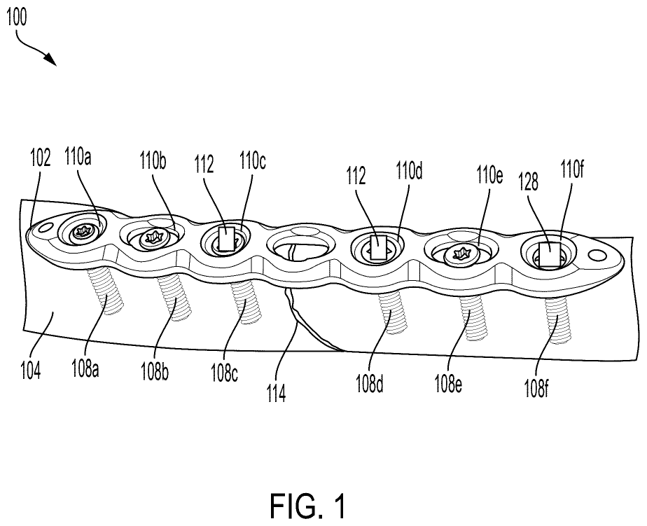

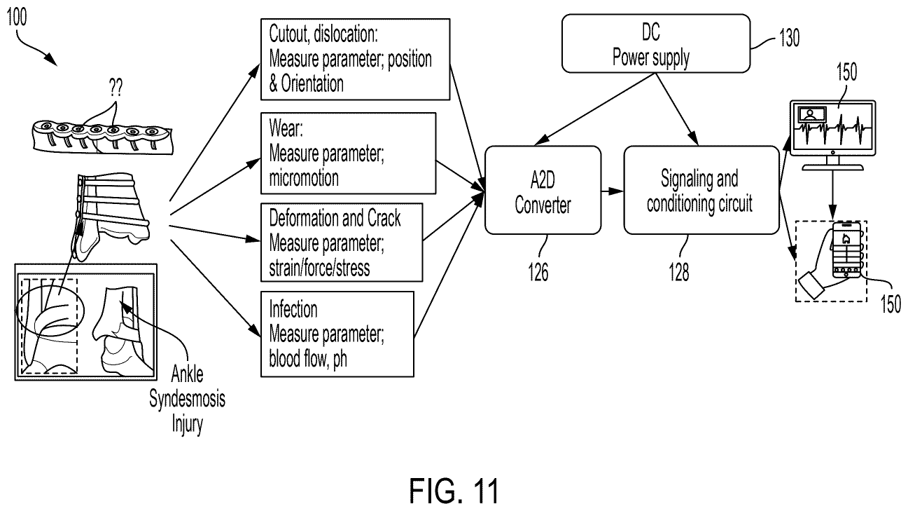

FIG. 1 is a perspective view of a bone plate system 100 according to an embodiment of the present disclosure. Bone plate system 100 includes a bone plate 102 positioned against a bone 104. Various bone screws 108a-108f are inserted through holes 110a-110f of bone plate 102 and are driven into bone 104. At least one of bone screws 108a-108f is configured as a “smart bone screw”, i.e., it houses an electronic component such as a sensor 112 that measures various conditions of bone plate 102, associated bone screw 108, and bone 104. An analog to digital converter 126 (FIG. 11) is configured to convert the analog sensor data to digital data. A processor 128 analyzes and stores the digital data, which is then transmitted to an external source 150 like a smart phone, tablet, monitor, network, etc. to allow for various monitoring of the condition of the bone plate as shown in FIG. 11.

Continuing with the embodiment of FIG. 1, bone plate 102 is configured to extend across multiple bone fragments separated by at least one fracture line 114 to promote compression of the bone fragments and faster healing. Bone plate 102 may be a tibial bone plate, femoral bone plate, or other types of bone plates adapted for various bones. Each of holes 110a-110f extends entirely through the thickness of bone plate 102 and may be a monoaxial hole or a polyaxial hole such that an operator can choose optimal positions for bone screws to be driven through bone plate 102 and into the underlying bone 104. Further, holes 110a-110f may be slotted or include various contouring around the perimeter of the holes to promote specific trajectories of bone screws. Such contouring may include ramps, scallops, and the like.

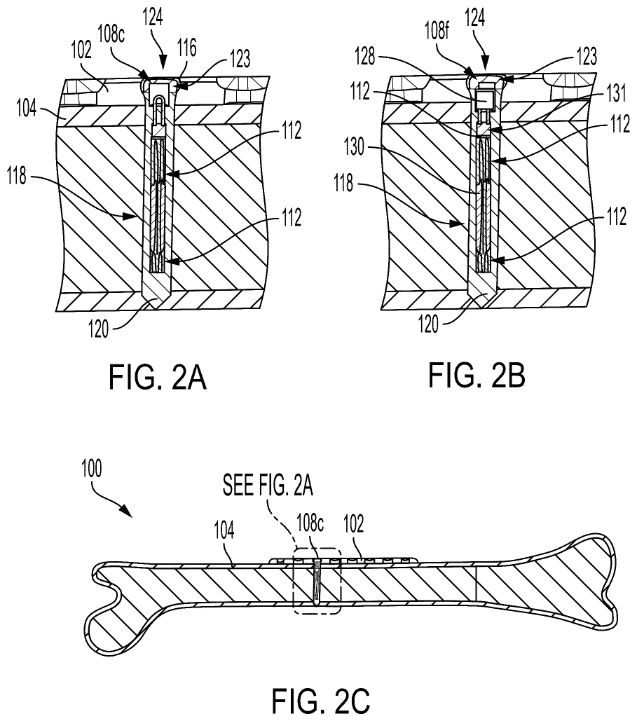

FIG. 2A illustrates a smart bone screw 108c that can be implemented with the various bone plate systems disclosed herein, such as bone plate system 100 illustrated in FIG. 2C. Notably, bone screw 108c may be inserted through any of holes 110a-110f of bone plate 102. For example, bone screw 108c may be inserted through hole 110c on a first side of fracture line 114 and bone screw 108d may be inserted through hole 110d on a second side of fracture line 114 such that various parameters may be measured on each side of fracture line 114. Additionally, certain bone screws within bone plate system 100 may be standard bone screws, i.e., screws that do not contain sensors or other electronic components. Such screws are illustrated in FIG. 1 as bone screws 108a, 108b, and 108e. Other bone screws implemented with bone plate system 100, such as bone screw 108f illustrated in FIG. 2B, may be a smart screw that includes additional electronic components, such as processor 128 and battery 130. Thus, it is foreseeable that any bone screw implemented with bone plate system 100 may be a smart screw, and the position of the smart screw is not limited to any particular hole.

Continuing with the embodiment of FIG. 2A, bone screw 108c includes a head 116, a shank 118, and a tip 120. A thread pattern 122 is defined around a perimeter of at least tip 120, but optionally around shank 118 as well. A central opening 124 configured to house a sensor 112 is defined along a longitudinal axis of bone screw 108c through shank 118 and at least partially through head 116. Central opening 124 may extend entirely through head 116, as illustrated in FIGS. 2A and 2B, such that it can be accessed by an operator, or it may extend partially through head 116 and only accessed by removing head 116. In either configuration, a hermetic seal (not shown) may be formed at the access opening of central opening 124 such that any components within central opening 124 are prevented from contacting the patient's tissue. As illustrated, central opening 124 has a diameter that is slightly larger than a corresponding diameter of a sensor 112 such that sensor 112 fits snuggly within opening 124 without becoming damaged due to potential movement of the screw. It is foreseeable that central opening 124 may be a non-cylindrical opening, and may be defined according to a particular sensor implemented with bone plate system 100. Bone screw head 116 may be configured as a bone screw cap 123 that is attachable to shank 118 via threads, snap fittings, and the like. Alternatively, the bone screw may be a monolithic bone screw with a central opening 124 extending out a proximal end of the bone screw, and a separate bone screw cap may be placed over the proximal end of the bone screw to hermetically seal the central opening 124. A removeable bone screw cap allows an operator to access central opening 124 and any electronic components within central opening 124 during an initial surgical procedure or during subsequent procedures, which is advantageous for the swapping and replacement of electronic components within central opening 124. Central opening 124 may extend entirely through or partially through the bone screw cap, or it may only extend through shank 118 and be sealed by the bone screw cap. It is foreseeable that bone screw 108c may be a headless screw or other screw types known in the art.

FIG. 2B illustrates a smart bone screw 108f according to an embodiment of the present disclosure. Bone screw 108f is substantially similar to bone screw 108c of FIG. 2A, and therefore only the differences will be described herein for sake of brevity. Bone screw 108 includes a processor 128 and a battery 130. Processor 128 is attached to a circuit board (not shown) and is powered by battery 130. Processor 128 may be any processor known in the art, and preferably includes an arithmetic and logical unit (ALU), a control unit such as a microcontroller, and a memory unit. Processor 128 is configured to communicate with and analyze and store digital data received from sensors 112. Such communication may be facilitated by wireless communication sources, such as Bluetooth, or from wired connections between different bone screws via an antenna 131. A sensor 112 may also be positioned within a central opening 124 of bone screw 108f. Any electronic components within bone screw 108f are hermetically sealed using screw caps 123 or similar sealing methods to those described for screw 108c above.

Smart screw 108c includes a sensor 112 that is cable of measuring a variety of parameters of bone plate system 100 and the surrounding bone 104. Additionally, the smart screws of other embodiments disclosed herein include similar sensors. Such sensors may be any one or combination of a variety of sensors including strain gauges, accelerometers, gyroscopes, temperature, piezoelectric, Hall effect sensors, micro-electro-mechanical systems (MEMS), ultrasonic, optic, pH, and the like. Such sensors are configured to measure a variety of parameters of the bone plate system and surrounding bone, and such parameters may be used to determine wear to the bone plate system and potential infections/conditions within the bone and surrounding tissue of the patient. For example, the sensors may be used to measure the force or pressure between a bone plate and a bone, and measure forces experienced within the bone plate. Based on such forces and based on further detection of movement of components within the bone plate system, the sensors may also measure the amount of wear of the bone plate system, which could be indicative of potential subsidence and/or infections. Additionally, the sensors may detect vibration, temperature, and other characteristics that may indicate implant wear or degradation over time. Such measurements and analysis are explained in further detail below. In other embodiments, the sensors can be configured to facilitate tissue healing. For instance, methods such as ultrasonic tissue stimulation, electrical stimulation, transcutaneous electrical nerve stimulation (TENS), infrared (IR) light therapy, etc., may be employed to facilitate the healing process. Additionally, small, programmable light-emitting diodes (LEDs) mounted on the device can emit light at various wavelengths to promote tissue regeneration in the affected area. In another embodiment, a miniature motor embedded within the smart screw can generate vibrations, serving as a means of stimulation to further aid in tissue repair. These components can be powered remotely, as disclosed herein, and activated for brief intervals according to a specified therapeutic protocol. Furthermore, the measurement of tissue impedance between the tips of different screws can be conducted to monitor and adapt the healing process effectively. The ability to measure tissue impedance can provide a valuable feedback mechanism, enabling real-time adjustments to the therapy based on the specific healing needs of the tissue. By analyzing the impedance data, the sensor can dynamically adjust the intensity and type of stimulation to specifically tailor the stages of the healing process.

In addition to or instead of relying on battery 130 to provide power to the electronic components within bone plate system 100, certain wireless charging methods may be implemented to power the various electronic components of the bone plate systems disclosed herein. It is appreciated that certain wireless charging method, such as near-field communication (NFC) or non-contact type charging may also be used for communication purposes. As such, an external device, such as a smart phone or other portable power supply, may be positioned adjacent the bone plate system and provide power to one or more electronic components therein when positioned a sufficient distance away from the electronic component. Alternative non-wired power transmission may also be achieved through induction or other wireless power transmission modes such as magnetic resonance, radio frequency energy transfer, capacitive coupling, ultrasonic power transfer, etc. Such wireless power transmission modes allow for “passive” monitoring of the bone plate system, meaning that they are not constantly sensing parameters of the bone plate system, and instead are only activated when desired. Such monitoring may extend the lifespan of the various electronic components within the smart bone plate systems when compared against battery-powered smart bone plate systems.

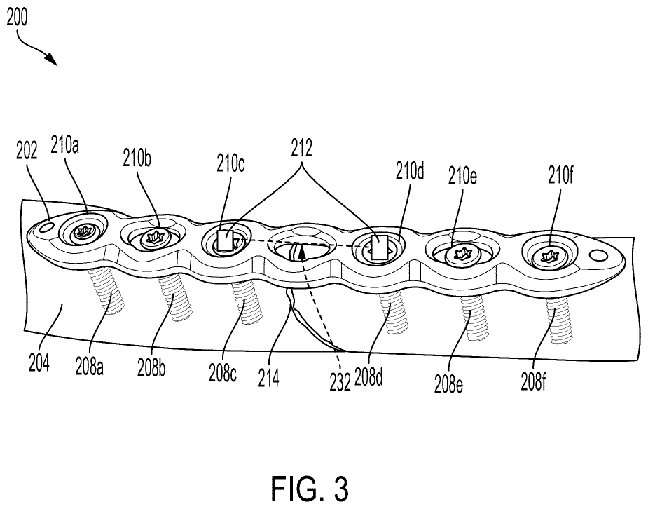

FIG. 3 illustrates another embodiment of a bone plate system. In this embodiment, bone plate system 200 is similar to bone plate system 100, and therefore like elements are referred to with similar numerals within the 200-series of numbers. Bone plate system 200 includes a bone plate 202 positioned against bone 204. Various bone screws 208a-208f are inserted through holes 210a-210f and are driven into the underlying bone 204. At least two of the bone screws, such as bone screws 208c and 208d, may include sensors 212 configured to monitor various parameters of the associated bone screws, bone plate, and surrounding bone. Bone screws 208c and 208d, or other bone screws within bone plate system 200 may further include a battery, processor, or other electronic components for analyzing, storing, and transmitting sensor data.

Continuing with the embodiment of FIG. 3, at least one wire-type sensor 232 is configured to extend between various screws 208a-208f and is configured to measure relative motions, deflection, and strain between various bone screws, which could be indicative of bone plate system wear or degradation of the patient's bone and/or surrounding tissue. Wire-type sensor 232 may be hermetically sealed within a protective casing to ensure the sensor does not contact the surrounding tissue. Alternatively, wire-type sensor 232 can be placed along a fully enclosed or partially enclosed channel extending through bone plate 202. It is appreciated that wire-type sensor 232 may be coil based and/or strip based. Additional sensor types that may be implemented with wire-type sensor 232 or as a separate sensor include strain gauges, piezoelectric sensors, motion sensors, resistance-based sensors, linear variable differential transformer (LVDT) sensors, optic sensors, diaphragm sensors, ultrasonic sensors, force-based sensors, thermal sensors, and the like.

Wire-type sensor 232 may be advantageous over using multiple smart screws because wire-type sensor 232 may be fully configured to detect parameters of bone plate system 200, process such data with an onboard processor, and communicate such data to an external source. Thus, the number of “smart” components within bone plate system 200 is reduced as compared to bone plate system 100. Additionally, strain may be measured along the length of wire-type sensor 232 as opposed to using individual strain gauges within smart screws, which simplifies the overall bone plate system 200. Using wire-type sensors, such as sensor 232, results in a reliable bone plate system as wire-type sensors directly replicate the effect of changes in the bone plate, bone, and any bone screws across the location of the fracture or at another desired location. Additionally, wire-type sensors can measure relative parameters (e.g., micromotions, force, displacement, strain, etc.) more efficiently as they include physical contacts. For example, a wire-type sensor may flex when a bone plate is flexing on a tension side or a compression side. In another embodiment of the present disclosure, the wire-type sensor may function as a screw head cover or cap. Each smart screw can include a terminal, for instance, within the hex drive feature, that can receive this cap. The cap, when attached to the smart screw, not only establishes an electrical connection but also helps to seal the top of the smart screw more effectively. The wire-type sensor could extend between this cap and one or more additional caps, facilitating the creation of a network of interconnected smart screws. Alternatively, a single monolithic cap could be utilized to cover and connect multiple smart screws, streamlining the setup and enhancing the overall structural integrity and connectivity of the system. In another embodiment, a wire instead of a wire-type sensor can be used to connect two or more smart screws to create a network. This wire can serve as a simple connection means between multiple smart screws to allow for communications between the connected smart screws.

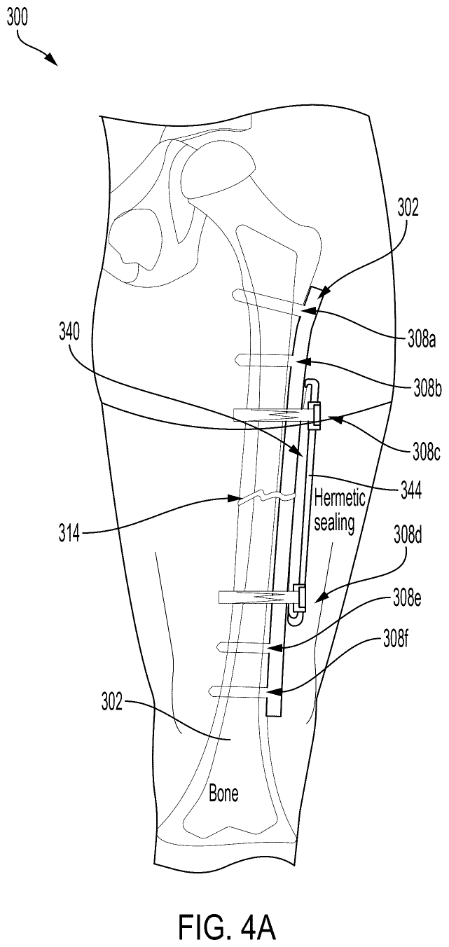

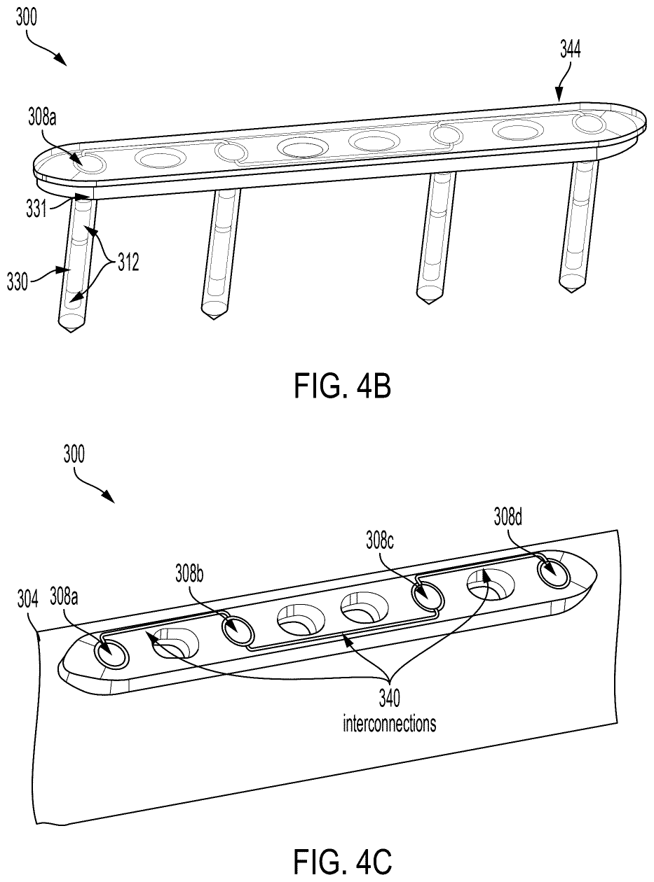

FIGS. 4A-4C illustrates another embodiment of a bone plate system. In this embodiment, bone plate system 300 is similar to bone plate system 100, and therefore like elements are referred to with similar numerals within the 300-series of numbers. Bone plate system 300 includes a bone plate 302 positioned against bone 304. Various bone screws 308a-308f are inserted through holes of bone plate 302 and are driven into bone 304. At least one of bone screws 308a-308f is configured as a “smart bone screw,” i.e., it includes a sensor 312 that measures various parameters of bone plate system 300 and the surrounding tissue, a power source 330, and an antenna 331. As illustrated in FIG. 4A, bone screws 308c and 308d are smart bone screws, and bone screws 308a, 308b, 308e, and 308f may be standard bone screws or bone screws that house other electronic components, such as processors, batteries, memory systems, and the like; however, alternative configurations may be implemented where different bone screws act as smart screws, such as the configurations in FIGS. 4B and 4C. Smart bone screws 308c, 308d are substantially similar to smart bone screws 108c, 108d, and therefore a detailed description of such screws is omitted for sake of brevity.

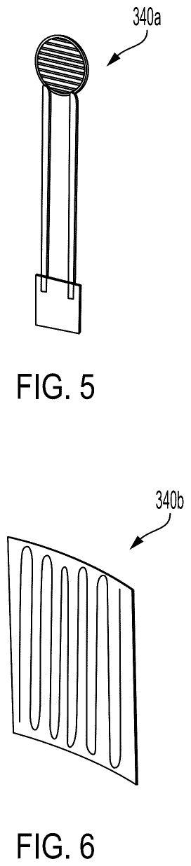

Bone plate system 300 includes a plate sensor 340 that extends longitudinally along a length of bone plate 302 such that it spans across at least one fracture line 314 of bone 304. Bone plate sensor 340 may be a wire-type sensor that extends across the bone plate and interconnects the various smart screws. While bone plate system 300 includes smart bone screws and a plate sensor 340 in this embodiment, other embodiments may include plate systems with only plate sensor and no screw sensors. Plate sensor 340 may be a foil sensor 340a (FIG. 5), diaphragm sensor 340b (FIG. 6), optic sensor, gap/clearance sensor, and the like. A hermetic sealing layer 344 may be placed over sensor 340 to protect sensor from the surrounding tissue. Alternatively, plate sensor 340 can be placed along a fully enclosed or partially enclosed channel extending through bone plate 302

Plate sensor 340 is configured to measure a variety of parameters relating to bone plate system 300 and bone 304, including flexing and stretching that occurs based on the loading of bone plate system 300. Additionally, it may measure bone bulge by measuring the load impinged on bone plate 302 by bone 304 or by measuring expansion and contraction of the bone or a gap between the bone and the bone plate via a diaphragm sensor or other sensor types. Plate sensor 340 may also measure stresses exerted on bone 304 by bone plate system 300 and vice versa, and such stresses may be indicative of proper/improper healing over a period of time. Diaphragm sensor 340b may also measure the frictional energy and static electricity between bone plate system 300 and bone 304. Ultrasonic and/or electromagnetic wave sensors may be used to measure a time difference and/or energy loss between the transmission and receival of such waves from the plate sensors, which may be indicative of proper/improper bone healing.

Plate sensor 340 is configured to be positioned either on an outer surface of bone plate 302, as is illustrated in FIG. 4A or an on inner surface of bone plate 302. Plate sensor 340 may include holes 342 configured to extend over a screw shank of various bone screws. Alternatively, plate sensor 340 may be attached directly to bone plate 302 without attaching to bone screws 308 by being positioned within a pocket of bone plate 302 and further be attached to bone plate 302 via screws. Plate sensor 340 can be optimized for a particular fracture by selecting a plate sensor 340 that is an appropriate size for the fracture. For example, a shorter sensor 340 may be selected for a fracture involving a single fracture line 314 and two bone fragments, and a longer sensor may be selected for a larger fracture involving multiple fracture lines and at least three bone fragments. Plate sensor 340 may be powered by a battery mounted within a smart bone screw, induction, NFC, and the like. If powered by induction, NFC, or other wireless power transmission methods, plate sensor 340 may be inactive when it is not in proximity to the power source and active when in proximity to the power source such that the lifespan of the sensor may be prolonged.

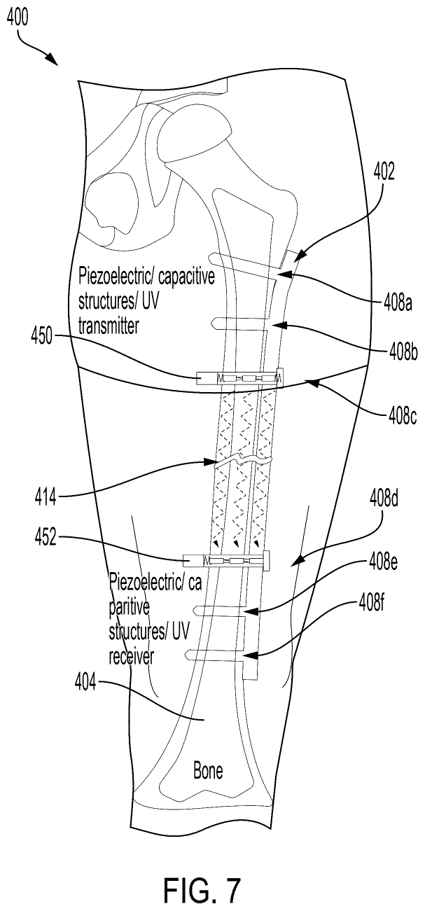

FIG. 7 illustrates another embodiment of a bone plate system. In this embodiment, bone plate system 400 is similar to bone plate system 100, and therefore like elements are referred to with similar numerals within the 400-series of numbers. Bone plate system 400 includes a bone plate 402 positioned against bone 404. Various bone screws 408a-408f are inserted through holes (not shown) of bone plate 402 and are driven into bone 404. At least one of bone screws 408a-408f is configured as a “smart bone screw,” i.e., it includes a sensor device that measures various parameters of bone plate system 400 and the surrounding tissue. As illustrated, bone screws 408c and 408d are smart bone screws, and bone screws 408a, 408b, 408e, and 408f may be standard bone screws or bone screws that house other electronic components, such as processors, batteries, memory systems, and the like.

Smart bone screws 408c, 408d are configured to send ultrasonic or electromagnetic waves to each other to detect condition of bone plate system 400 and bone 404. As such, one of smart bone screws, such as screw 408c, may include a transmitter 450 configured to send an ultrasonic or electromagnetic wave towards screw 408d. Screw 408d includes a receiver 452 configured to receive the wave transmitted from transmitter 450 and communicate with a processor either onboard with bone plate system 400 or external to bone plate system 400. As such, transmitter may communicate via wired transmission, Bluetooth, or other data communication methods. Likewise, smart screws 408c, 408d may be powered via wired connection to a battery, induction, NFC, and the like.

The specific position of the smart bone screws may be optimized for a particular fracture. For example, as illustrated in FIG. 7, each of bone screws 408c, 408d is positioned on opposing sides of fracture line 414. As such, the transmitted waves pass through fracture line 414, and are able to monitor the condition of fracture line 414 over a period of time. A skilled artisan would appreciate the ability to implement smart screws in particular bone holes of bone plate 402 based on particular fractures. It is also foreseeable that smart screws, such as bone screws 408c, 408d may be implemented in direct healing methods where plates and/or implants are not implemented with a patient. As such, smart bone screws having a transmitter and a receiver may be drilled into a bone on opposing sides of a fracture line without being secured to any type of plate and/or implant. The smart bone screws may then monitor the bone condition over time similar to the smart bone screws 408c, 408d of bone plate system 400.

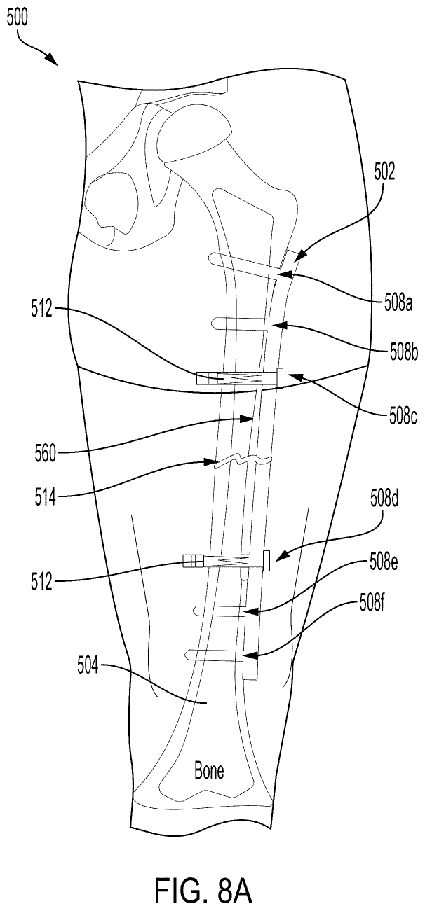

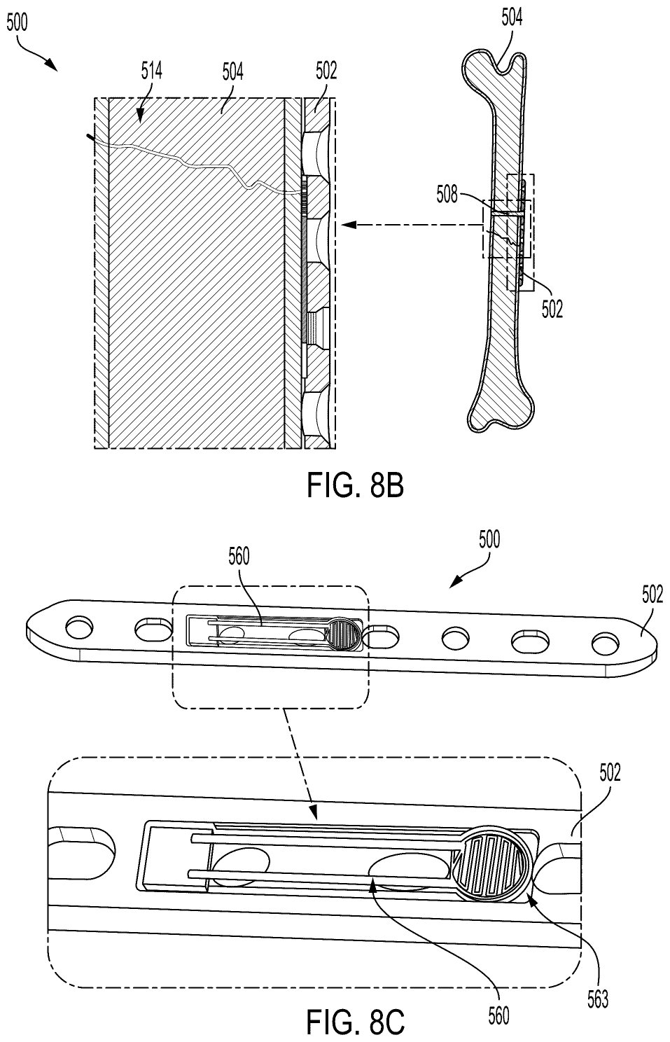

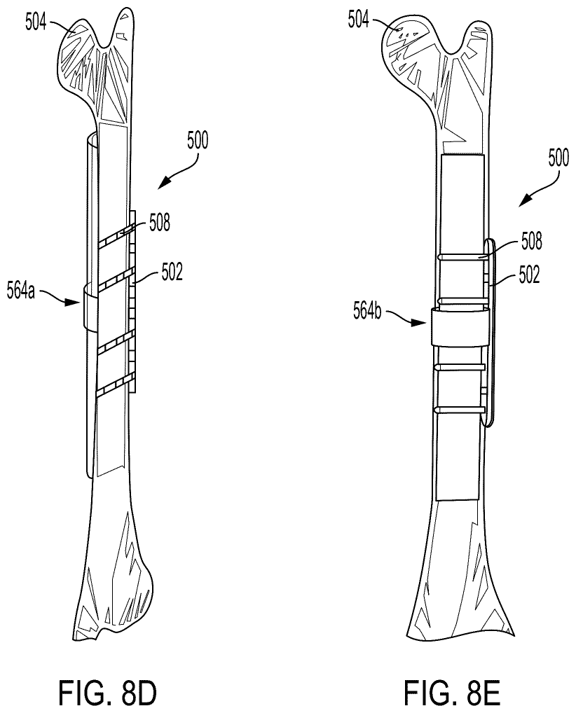

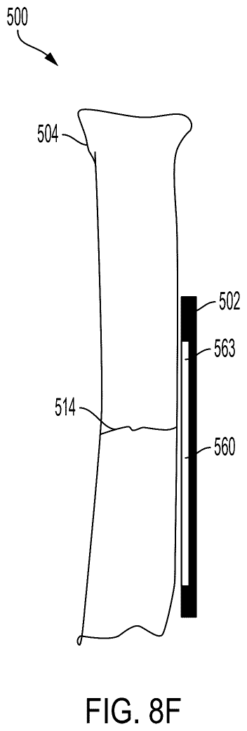

FIGS. 8A-8E illustrate another embodiment of a bone plate system. In this embodiment, bone plate system 500 is similar to bone plate system 100, and therefore like elements are referred to with similar numerals within the 500-series of numbers. Bone plate system 500 includes a bone plate 502 positioned against bone 504. Various bone screws 508a-508f are inserted through holes (not shown) of bone plate 502 and are driven into bone 504. At least one of bone screws 508a-508f is configured as a “smart bone screw,” i.e., it includes a sensor 512 that measures various parameters of bone plate system 500 and the surrounding tissue. As illustrated, bone screws 508c and 508d are smart bone screws, and bone screws 508a, 508b, 508e, and 508f may be standard bone screws or bone screws that house other electronic components, such as processors, batteries, memory systems, and the like. Smart bone screws 508c, 508d may be substantially similar to smart bone screws 108c, 108d such that they sense various parameters of bone plate system 500 and bone 504, or may be substantially similar to smart bone screws 408c, 408d such that they contain a transmitter and receiver and detect changes in ultrasonic and/or electromagnetic waves over time.

Bone plate system 500 further includes a pressure mapping sensor 560 (illustrated in FIGS. 8B and 8C) positioned between bone plate 502 and bone 504. Pressure mapping sensor 560 is elongated and is configured to extend across multiple bone fragments defined by at least one fracture line 514. Pressure mapping sensor 560 is particularly configured to detect pressure imparted on bone plate 502 from calluses that form on bone 504 over a period of time after a surgical procedure. As calluses are generally expected post-procedure, an absence of pressure detected underneath bone plate 502 may be indicative of improper healing. Pressure mapping sensor 560 may also be partially recessed within an undercut 563 bone plate 502, as is illustrated in FIG. 8C and FIG. 8F. Such an under cut 563 allows pressure mapping sensor 560 to sit below or flush with an upper surface of bone plate 502 such that it does not catch or improperly engage surrounding tissue. Alternatively, the pressure mapping sensor may be configured as an at least partially cylindrical pressure sensor that wraps around at least a portion of the bone. For example, pressure mapping sensor 564a of FIG. 8D may wrap partially around bone 504 and pressure mapping sensor 564b of FIG. 8E may wrap entirely around bone 504. Each of pressure mapping sensors 564a, 564b may communicate with the various smart screws of bone plate system 500 similar to the other embodiments disclosed herein. Further, pressure mapping sensors 564a, 564b are configured to measure hoop stress and/or radial stresses imparted on the bone due to hematoma formation or fracture healing, each of which can be indicative of overall patient health.

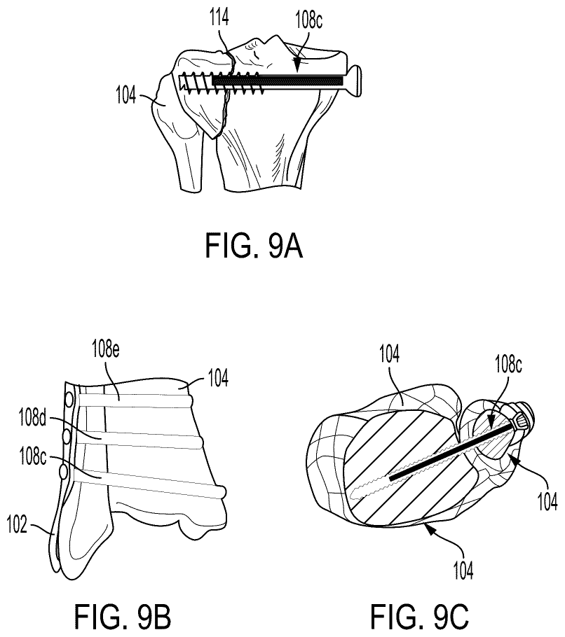

FIGS. 9A-9C illustrate embodiments of a smart screw 108 that may be used independently of a bone plate system, i.e., can be used to secure a fractured bone with or without the use of a bone plate. For example, FIG. 9A illustrates a hermetically sealed smart screw 108c similar to the smart screw illustrated in FIG. 2A. Such a smart screw is configured to be driven through fragments of a bone 104 separated by a fracture line 114. FIG. 9B illustrates three hermetically sealed smart screws 108c, 108d, 108e that are driven through a bone plate 102. FIG. 9C illustrates a hermetically sealed smart screw 108c configured to be driven into a bone. Such a smart screw acts as a syndesmosis screw and is indicative to an operator of bone healing based on factors such as load distribution at different sides of a bone join, soft tissue healing, weakening of bone screw 108c, and the like. It should be understood that any of the smart screws described herein, including smart screw 108, can function independently of a bone plate system. These screws are versatile and can be applied in various medical procedures, such as treating femoral neck fractures or facilitating ankle fusions for example, without necessitating a bone plate system. For instance, in the treatment of a femoral neck fracture, the smart screw can be strategically positioned to traverse the fracture line. Sensors integrated into either side of the smart screw enable continuous tracking and monitoring of the healing process, thereby providing valuable real-time data on the patient's recovery. This data allows physicians to make informed decisions about further treatment options and adjustments to ensure optimal healing outcomes. While some embodiments disclosed herein feature a hermetically sealed smart screw, other embodiments may incorporate smart screws that lack hermetic sealing. For instance, a smart screw equipped with a pH sensor may not necessitate a hermetic seal, as the absence of such sealing allows the sensor to directly contact patient tissue. This direct contact will allow for accurate recording and collection of pH data from the surrounding biological environment, thereby enhancing the diagnostic capabilities of the device.

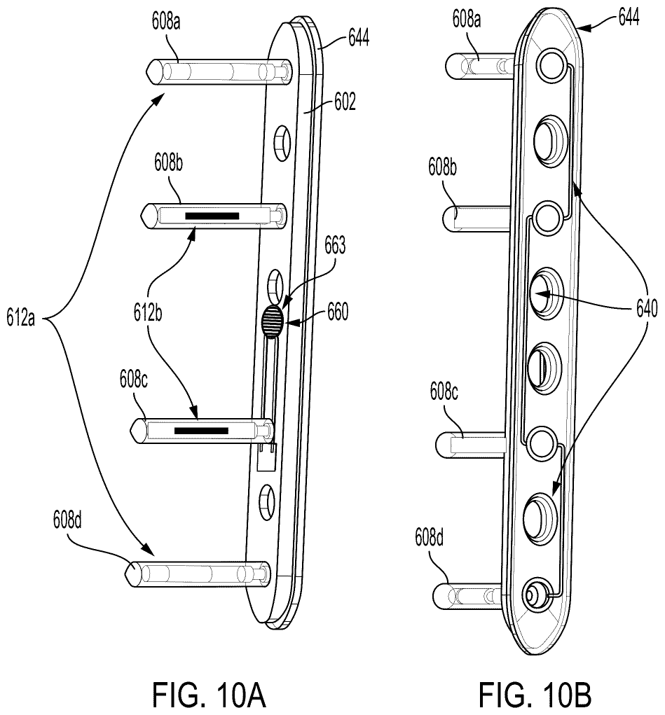

FIGS. 10A and 10B illustrate another embodiment of a bone plate system. In this embodiment, bone plate system 600 is similar to bone plate system 100, and therefore like elements are referred to with similar numerals within the 600-series of numbers. It is appreciated that an operator may implement any combination of sensors disclosed herein in a single surgical procedure as different sensor types provide different information regarding bone healing. For example, a pressure mapping sensor may be more accurate in predicting if initial stages of bone healing (e.g., hematoma formation) is occurring, whereas ultrasonic sensors or strain-based sensors may be more accurate in predicting the cortical cell formation indicative of later stages of bone healing. The operator and/or patient may choose which sensor data they desire to receive. Particularly, FIG. 10A illustrates a bone plate system 600 implementing both strain gauge sensors 612a in smart screws 608a, 608d and at least one of a piezoelectric, capacitive, UV, or other sensor type 612b disclosed herein in smart screws 608b, 608c. Additionally, a pressure mapping sensor 660 may be positioned within an undercut 663 of bone plate 602 and a hermetic sealing layer 644 is positioned over a top of bone plate 602. FIG. 10B illustrates a bone plate system 600 with a similar sensor configuration to the bone plate of FIG. 10A, albeit with an additional hermetic sealing layer 644 and an interconnected bone plate sensor 640.

FIG. 11 illustrates a schematic flowchart of a method of using the various bone plate systems disclosed herein, particular bone plate system 100. The method of using bone plate system 100 will be described in detail, and then the differences in the other bone plate systems disclosed herein will be described after. To implant bone plate system 100, an operator may first assess the patient and determine a particular bone plate configuration that is best suited for the particular fracture. The patient may then undergo a surgical procedure to implant a bone plate 102 over a fractured bone 104.

An operator may then insert various bone screws 108 through holes 110 of bone plate 102 into the underlying bone 104. An operator may select certain screws as being smart screws, such as screws 108c, 108d as illustrated in FIG. 1, and insert them into bone plate 102 on opposing sides of fracture line 114. It is important to note that when assessing the fractured bone, an operator may select any number of screws as being smart screws, regardless of the screws' position in the bone plate. An operator may also assess whether the selected smart screws are to be battery operated or operated through wireless methods, such as induction-based power transfer methods. If the smart screws are to be powered via battery, an operator may select a secondary screw, such as screw 108f as illustrated in FIG. 1, as a screw that contains a battery, processor, circuit board, memory, and the like. Before the screws are inserted into the bone plate, an operator may remove the screw cap on the smart screws and insert sensors, such as sensor 112, into the central opening 124 of the smart screws. Once closed, such screw caps provide a hermetic seal that allows the sensors contained therein to be safely used.

An operator may then use a series of targeting guides, aiming blocks, and the like to ensure accurate alignment and placement while inserting the selected bone screws. Once the screws are inserted, an operator may again remove the various screw caps to access the electronic components within the bone screws. Such modularity is also beneficial if revision surgeries are ever required or if the electronic components within the smart screws need to be serviced and/or replaced. If a battery is used to power the electronic components within the smart screws, then an operator can ensure that each component is properly attached to the battery and that the processor is capable of communicating with an external device, such as a smart phone, tablet, or computer. If NFC or a similar wireless power transmission method is used to power the electronic components within the smart screws, then an operator may hold a powered device near bone plate system 100 to provide power to the various electronic components.

Once the initial surgery is completed, data can be collected to obtain threshold parameters as sensed by sensors 112. As sensors 112 sense various parameters, they communicate those parameters to processor 128 via analog to digital converter 126. Processor 128 may then store such parameters and communicate them to an external source 150, such as a smart phone, tablet, or computer. Changes in parameters over time may be indicative of improper healing and may indicate to an operator that further investigation is required to analyze the bone plate system.

Bone plate system 200 may be implemented similarly to bone plate system 100, albeit an operator may ensure that a wire-type sensor 232 is placed between particular smart screws. Wire-type sensor 232 is configured to collect additionally data over the sensors 212 positioned within various smart screws of bone plate system 200, and wire-type sensor 232 communicates such data to a processor via an analog to digital converter. Bone plate system 300 may be implemented similarly to bone plate system 100, albeit an operator may ensure that a sensor 340, such as a foil sensor or diaphragm sensor, is positioned against an inner side of bone plate 302 before mounting bone plate 302 to bone 304 or on an outer side of bone plate 302 after mounting bone plate 302. A hermetic sealing layer 344 is also positioned over the sensor 340 to ensure sensor 340 is properly sealed from the surrounding tissue. Sensor 340 may then communicate with a processor via an analog to digital converter. Bone plate system 400 may be implemented similarly to bone plate system 100. The sensors 412 of bone plate system 400 may be placed in smart screws on opposing sides of a fracture line of a bone. The sensors 412 are configured as transducers and receivers, and may communicate with a processor via an analog to digital converter. Over a period of time, the sensors can detect various changes in the received ultraviolet and/or electromagnetic waves from initial threshold values that were measured after implantation. If the change over time grows over an expected value, it may be indicative of improper bone healing, infection, subsidence, or other medical abnormalities. Bone plate system 500 may be implemented similarly to bone plate system 100. Bone plate system 500 further includes a pressure mapping sensor 560 that is positioned between bone plate 502 and bone 504. Pressure mapping sensor 560 is configured to measure the pressure exerted on bone plate 502 from bone 504, and communicate such pressure values to a processor via an analog to digital converter.

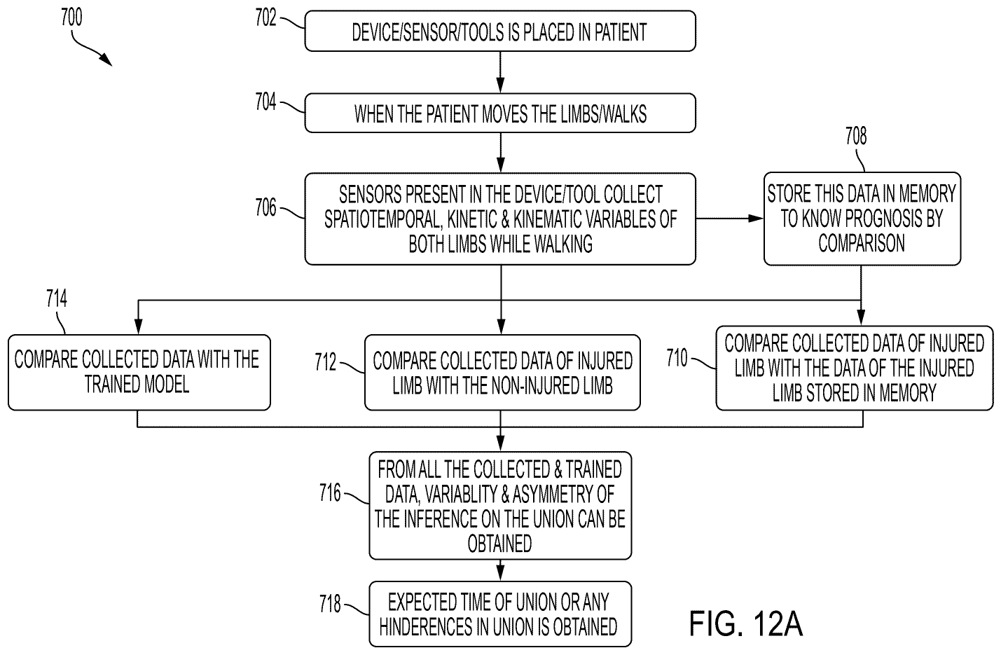

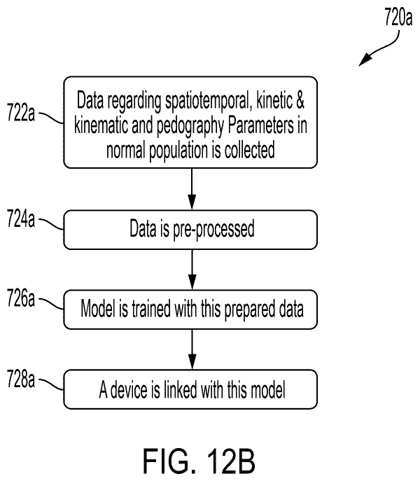

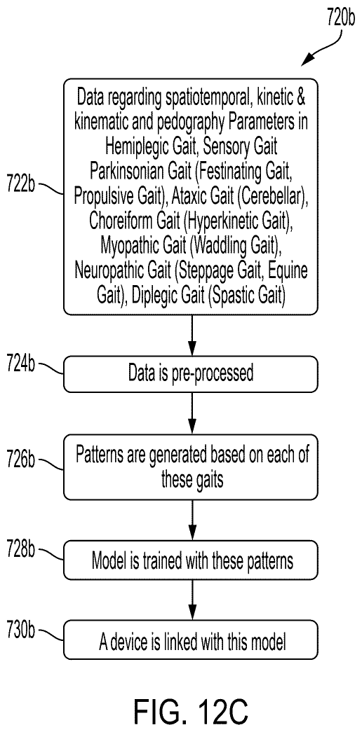

The bone plate systems disclosed herein may implement artificial intelligence (AI) technology to aid a patient in the healing process. Particularly, a first AI system 700 illustrated by the flowchart of FIG. 12A may monitor a patient's walking patterns over a period of time after implantation of a bone plate system. This may be accomplished by the smart screws of the bone plate system communication with an external device, such as a smart phone, smart watch, and the like that can be wearable on the patient's body. The external device, in conjunction with the sensors of the bone plate system, may track movement pattern, inclination, range of motion, and the like. Over a period of time, AI system 700 can be trained to predict early symptoms of various conditions, such as arthritis of the knee, implant subsidence, Alzheimer's, and the like. AI system 700 can also compare other health parameters of a patient not measured from the bone plate system such as heart rate, blood pressure, and track those values with the values measured by the various sensors of the bone plate system.

The implementation of AI in this context offers significant benefits by enabling a proactive approach to the healing process. By continuously monitoring and analyzing patient data, AI system 700 can identify subtle changes or trends that may indicate the onset of complications or the need for adjustments in treatment. This early detection is crucial for preventing conditions from deteriorating, potentially reducing the need for additional surgeries or prolonged treatment regimens. Additionally, the AI's capacity to learn from data gathered across numerous cases allows for the development of optimized, patient-specific treatment plans. This data can guide decisions regarding the type of hardware fixation required and the precise placement locations that would best suit individual fracture patterns and biomechanical needs. Moreover, by aggregating and analyzing data from multiple patients over time, AI system 700 can refine its predictive algorithms, improving its accuracy in forecasting outcomes and necessary interventions. This continuous learning process enables the AI to update and adapt therapeutic plans dynamically, adjusting to each patient's healing progress and changing conditions. Ultimately, this leads to more personalized, efficient, and effective treatments, enhancing patient outcomes and optimizing the use of medical resources. Thus, AI system 700 provides the ability to customize therapy based on comprehensive real-time and historical.

Continuing with the embodiment of FIG. 12A, AI system 700 has an initial step 702 of inserting a medical device into a patient. Step 702 may entail inserting a bone plate system, such as bone plate system 100, into a patient. A second step 704 involves the patient moving after the surgical procedure to obtain initial data points. A third step 706 includes sensing various parameters of the medical device inserted into the patient and the surrounding bone/tissue via the sensors within the medical device. These sensors may collect any one or combination of spatiotemporal, kinetic, and kinematic variables of the patient while the patient is moving. From the third step 706, AI system 700 branches and allows for multiple options. One such option includes step 708, in which the data obtained in step 706 is stored in a memory system. Once stored, a later step 710 includes comparing the collected and stored data of the patient with a data stored in the memory system to determine a change in the patient's health parameters over a period of time. Another potential branch from step 706 is step 712, which involves comparing collected data from the patient with other data from the same patient. For example, if a bone plate system was implanted into a patient's left femur, the patient's health parameters pertaining to the left femur may be compared with the patient's otherwise healthy right femur. Another potential branch from step 706 is step 714, which includes comparing collected data with the trained model (explained below in reference to FIGS. 12B and 12C). After at least one of steps 710, 712, and 714 has been completed, a later step 716 results in obtaining an inference from the collected and trained data. A final step 718 includes creating an expected timeframe for further healing to occur or for additional scheduling additional medical procedures.

FIG. 12B illustrates a first data training method 720a. Data training method 720 has an initial step 722a of collecting data regarding spatiotemporal, kinetic, kinematic, and pedography parameters in a normal population. Such data can relate to a pace of a patient's walk, particularly the gate speed and stride length. Additionally, the data can relate to a rhythm of the patients walk, including the cadence, step time, single limb support time, stance time, and swing time. The data can also relate to a vertical acceleration signal, a range of movement of all joins in lower limbs, plantar flexor movement immediately following heel contact, power generated, moments, and angular velocity changes in a limb, plantar pressure variation and shift of center of pressure, muscle volume, ability to bear full body weight, maximum knee flexion and plantar flexion, ankle plantar flexion during pre-swing and hip extension during stance, and knee flexion during loading phases and ankle dorsiflexion during stance. A second step 724a includes pre-processing the data from step 722a. A third step 726a includes training a model with the pre-processed data from step 724a. A final step 728a includes linking an external device with the model of step 726a.

FIG. 12C illustrates a second data training method 720b similar to first data training method 720a. A first step 722b of training method 720b includes obtaining data regarding a patient's spatiotemporal, kinetic, kinematic, and pedography parameters in a hemiplegic gait, sensory gait, parkinsonian gait (festinating gait, propulsive gait), ataxic gate (cerebellar), choreiform gait (hyperkinetic gait), myopathic gait (waddling gait), neuropathic gait (steppage gait, equine gate), and diplegic gait (spastic gait). Notably, the spatiotemporal, kinetic, kinematic, and pedography parameters may be similar to the parameters disclosed in step 722a. A second step 724b includes pre-processing the data of step 724b. A third step 726b includes generating patterns based on each of the gaits of step 722b. A fourth step 728b includes training a model with such patterns. A final step 730b includes linking an external device with the model of step 728b.

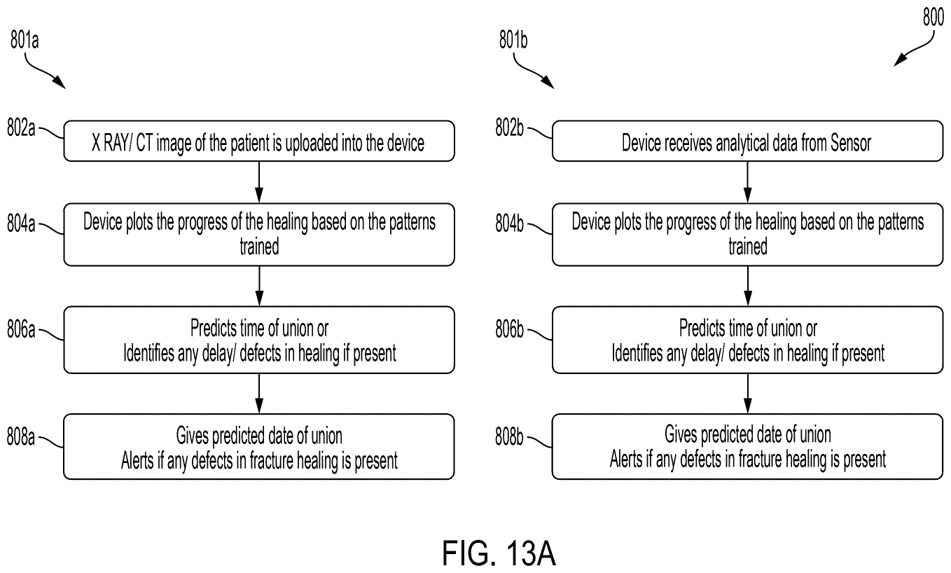

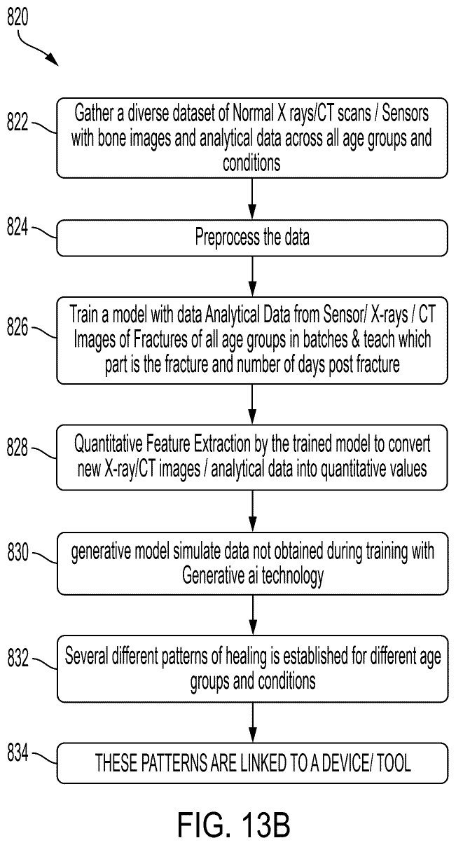

FIG. 13A illustrates a second AI system 800 including a first path 801a and a second path 801b. First path 801a begins with a first step 802a of uploading an X-ray or CT image of a patient to a device. A second step 804a includes plotting the progress of healing based on trained patterns. A third step 806a includes predicting the time of union and/or identifying any defects or delays hindering the healing process. A final step 808a includes predicting a union timeframe or creating alerts if a hindrance to creating a union is detected. Second path 801b includes a first step 802b of receiving analytical data from a sensor of a bone plate system. A second step 804b includes plotting the progress of healing based on trained patterns. A third step 806b includes predicting the time of union and/or identifying any defects or delays hindering the healing process. A final step includes a predicting a union timeframe or creating alerts if a hindrance to creating a union is detected.

FIG. 13B illustrates a data training method 820. Data training method 820 includes a first step 822 of gathering a diverse dataset of normal X-ray and/or CT scans and/or data from sensors compared with bone images, and additional analytical data across all age groups and conditions. A second step 824 includes pre-processing the data. A third step 826 includes training a model with data analytical data from the sensor data and/or X-ray and/or CT images of fractures of all age groups. Such data can be placed in batched to teach elements of the fracture and the timespan wince the fracture occurred. A fourth step 828 includes using the trained model to convert new X-ray and/or CT images and/or analytical data into quantitative values. A fifth step 830 includes simulating a generative model not obtained during training with generative AI technology. A sixth step 832 includes establishing several different healing patterns for different patient parameters such as age, height, etc. and different bone parameters such as type of fracture or medical condition. A final step 834 includes linking the patterns of step 832 to an external device.

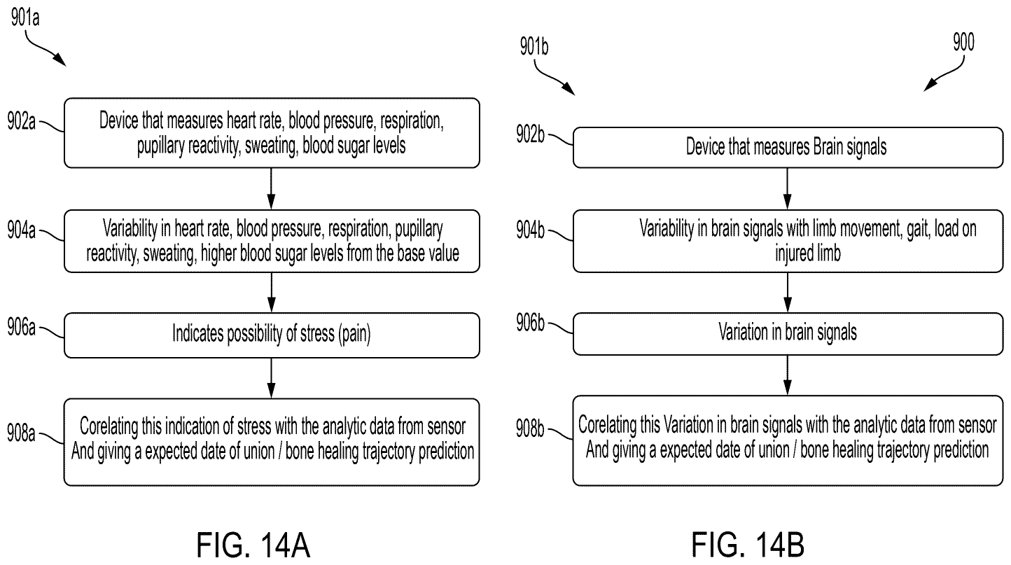

FIGS. 14A and 14B illustrate additional embodiments for implementing an AI system 901a and an AI system 901b. AI system 901a illustrated in FIG. 14A includes a first step 902a of obtaining a device that measures at least one of heart rate, blood pressure, respiration, pupillary reactivity, sweat, and blood sugar levels. A second step 904a includes monitoring a patient over a period of time until at least one of a variability in heart rate, blood pressure, respiration, pupillary reactivity, sweat, and blood sugar levels is detected to have changed compared to a base value obtained in step 902a. A third step 906a includes indicating a possibility of stress concentrations based on the difference obtained in step 904a. Such a stress concentration may be indicative of pain, implant shifting, disease, poor bone condition, and the like. A final step 908a includes corelating an indication of stress concentration with analytic data from a sensor of a bone plate system and giving an expected date of union and/or establishing a recovery timeframe.

AI system 901b illustrated in FIG. 14B includes a first step 902b of obtaining a device that measures brain signals. A second step 904b includes monitoring a patent over a period of time until at least one of a variability in brain signals with limb movement, gait, and load on an injured limb is detected to have changed compared to a base value obtained in step 902b. A third step 906b includes indicated variation of brain signals of step 904b. A final step 908b includes corelating the variation of step 906b with analytic data from sensors of a bone plate system and giving an expected date of union and/or establishing a recovery timeframe. Each of the bone plate systems disclosed herein may be operated similarly to bone plate system 100. Each system can measure threshold parameters via any sensors, compare the measured parameters against stored parameters, and determine a change in parameters over time. If a change in parameter values exceeds an expected value, it may be indicative of a larger issue, such as implant loosening, subsidence, infection, improper callus formation, and the like.

A kit containing the various components of bone plate systems 100-500 is disclosed herein. For example, a kit may include a bone plate 102, various bone screws 108 (both smart screws and traditional screws), sensors, batteries, processors, screw caps, and an external device. Further, each component may be included in a plurality of difference sizes to allow an operator to select a proper size for each implementation. The kit may further include various instrumentation for implanting the bone plate systems, e.g., aiming guides, retractors, etc.

While a bone plate system for a long bone such as a tibia is described above, all or any of the aspects of the present disclosure can be used with any other bone plates adapted for other bones, such as the femur, humerus, and the like. Additionally, all or any of the aspects of the present disclosure may be implemented with various implants, such as spinal implants, hip implants, knee implants, and the like. Sensor shape, size, and configuration can be customized based on the type of implant and patient-specific needs. Further, the components of the various embodiments disclosed herein may be substituted between embodiments.

Furthermore, although the invention disclosed herein has been described with reference to particular features, it is to be understood that these features are merely illustrative of the principles and applications of the present invention. It is therefore to be understood that numerous modifications, including changes in the sizes of the various features described herein, may be made to the illustrative embodiments and that other arrangements may be devised without departing from the spirit and scope of the present invention. In this regard, the present invention encompasses numerous additional features in addition to those specific features set forth in the paragraphs below. Moreover, the foregoing disclosure should be taken by way of illustration rather than by way of limitation as the present invention is defined in the examples of the numbered paragraphs, which describe features in accordance with various embodiments of the invention, set forth in the paragraphs below.

1. A fixation system comprising:
a bone plate configured to be placed against a bone;
a first bone screw insertable through a hole in the bone plate, the bone screw including a sensor hermetically sealed within the bone screw and configured to detect a characteristic of at least one of the system and the bone;
a power source configured to provide power to the sensor; and
a processor configured to output the characteristic to an external source.

2. The system of claim 1, further comprising a second bone screw insertable through a second hole in the bone plate having a second sensor hermetically sealed within the second bone screw, the second sensor configured to detect a second characteristic of at least one of the bone and the bone screw.

3. The system of claim 2, further comprising a wire sensor positioned between the first and second bone screws, the wire sensor configured to communicate with the processor to detect a third characteristic of at least one of the bone and the first and second bone screws.

4. The system of claim 2, wherein the power source is mounted within at least one of the first and second bone screws.

5. The system of claim 2, wherein the first and second bone screws are configured to be secured on opposite sides of a fracture line of the bone.

6. The system of claim 1, further comprising a third bone screw insertable through a third hole in the bone plate, wherein the power source is mountable within a body of the third screw.

7. The system of claim 1, wherein the power source is a battery.

8. The system of claim 1, wherein the power source is an external device capable of providing near field communication to at least one of the sensor and the processor.

9. The system of claim 1, wherein the sensor is at least one of a strain gauge, accelerometer, gyroscope, temperature sensor, piezoelectric sensor, hall sensor, micro-electro-mechanical systems sensor, ultrasonic sensor, and pH sensor.

10. The system of claim 1, wherein the characteristic is at least one of a vibrational measurement of the screw, force between the bone plate and the bone, temperature, acceleration, and friction of an interface defined between the bone and the system.

11. A method of securing a bone, the method comprising:
placing a bone plate adjacent the bone;
inserting a bone screw through a hole in the bone plate such that a sensor associated with the bone screw is configured to detect a characteristic; and
outputting information of the characteristic to an external source.

12. The method of claim 11, further comprising placing the sensor within a shank of the bone screw.

13. The method of claim 11, further comprising driving a second bone screw through a second hole in the bone plate.

14. The method of claim 13, further comprising powering the sensor with a battery housed within the second bone screw.

15. The method of claim 11, further comprising powering the sensor with an external source, the external source capable of providing near field communication to the sensor and a processor associated with the bone screw.

16. The method of claim 11, further comprising storing the characteristic in a memory.

17. The method of claim 11, wherein the outputting step includes a processor receiving the characteristic and converting it to a characteristic signature, the characteristic signature being at least one of a vibration data, movement data, force data, strain data, and temperature data.

18. A fracture fixation system comprising:
a bone plate configured to be placed against a bone;
a bone screw insertable through a hole in the bone plate, the bone screw associated with a processor; and
a pressure sensor configured to be positioned on at least one of an upper and lower side of the bone plate, the pressure sensor in communication with the processor and configured to detect pressure profiles of at least one of the bone and the system such that the processor can receive the pressure profiles and output the pressure profiles to an external source.

19. The system of claim 18, wherein the pressure sensor is a piezoelectric pressure sensor.

20. The system of claim 18, wherein the pressure sensor communicates with the processor by wireless communication.
